(19) United States
(12) Patent Application Publication
(10) Pub. No.: US 2010/0029985 A1
(43) Pub. Date: Feb. 4, 2010

(54) Process

(75) Inventors: George Robert Hodges, Bedfordshire (GB); Juliette Martin, Angers (FR); Noel Anthony Hammil, West Yorkshire (GB); Ian Nicholas Houson, West Yorkshire (GB)

Correspondence Address: Sunstein Kann Murphy & Timbers LLP, 125 SUMMER STREET, BOSTON, MA, 02110-1618, US

(73) Assignee: AVECIA PHARMACEUTICALS LIMITED, Blackley, Manchester (GB)

(21) Appl. No.: 11/722,454

(22) PCT Filed: Dec. 14, 2005
(86) PCT No.: PCT/GB05/04882

§ 371 Date: Mar. 11, 2008

(30) Foreign Application Priority Data
Dec. 22, 2004 (GB) 0428128.3

(51) Int. Cl. C07C 211/27; C07C 37/00

(52) U.S. Cl. 564/305; 568/814

(57) ABSTRACT

A process for the preparation of a compound of Formula (1) wherein Ar represents an optionally substituted hydrocarbyl or an optionally substituted heterocyclyl group comprising an aromatic moiety; and R¹ and R² each independently represent an optionally substituted hydrocarbyl or an optionally substituted heterocyclyl group; said process comprising: a) reducing a compound of Formula (2) to form a compound of Formula (3): b) activating the compound of Formula (3) to form a compound of Formula (4): wherein OX represents a leaving group; and c) coupling the compound of Formula (4) to a compound of Formula (5): to form a compound of Formula (1). A stereoselective reduction of ketones to alcohols is also disclosed.

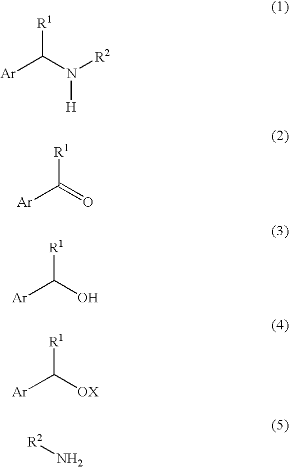

The present invention concerns a process for the preparation of secondary amines attached to a secondary carbon centre, particularly N-substituted benzylamines. We have found a process in which reduction and displacement can be achieved whilst substantially preserving the enantiomeric excess achieved. A further advantage is that all three steps of process of the invention can be conducted without the need to isolate the products of the intermediate steps.

U.S. Pat. No. 6,391,865 and J R Tagart et al, J. Med. Chem., 44, 3343 (2001) and WO 00/66558 disclose processes in which an alcohol is activated by a mesylate group and then displaced subsequently by an amine. However a drop of 20 to 40% in the enantiomeric excess is typically observed in the displacement step.

According to the present invention, there is provided a process for the preparation of a compound of Formula 1:

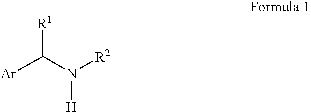

wherein

Ar represents an optionally substituted hydrocarbyl or an optionally substituted heterocyclyl group comprising an aromatic moiety; and

R¹ and R² each independently represent an optionally substituted hydrocarbyl or an optionally substituted heterocyclyl group;

said process comprising:
a) reducing a compound of Formula 2 to form a compound of Formula 3:

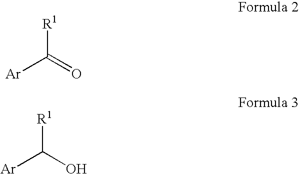

b) activating the compound of Formula 3 to form a compound of Formula 4:

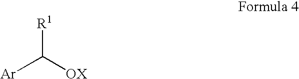

wherein OX represents a leaving group; and
c) coupling the compound of Formula 4 to a compound of Formula 5:

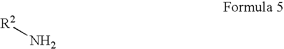

to form a compound of Formula 1.

Surprisingly we have found that there is a very small drop in the enantiomeric excess in the displacement step when reacting a compound of formula (4) with the compound of formula (5). This is so despite the presence of a good leaving group which would ordinarily promote some SN1 displacement to occur in addition to the SN2 displacement, leading to some racemisation and hence degradation in the enantiomeric excess obtained.

The control of the stereochemistry i.e. the preservation of the enantiomeric excess in the process of the present invention occurs regardless of the stereochemistry of the activated alcohol derivative of formula (4). In other words the control exists for both R and S enantiomeric forms.

The reactions may be carried out in discrete steps with the products being isolated at each step or one or more steps can be carried out without isolation of the intermediate products. Thus the sequence of reactions can be performed as a ‘one pot’ procedure. The ‘one pot’ procedure is preferred on the basis of ease of conducting the process. Waste solvents and other waste materials are minimised as is the need for handling since a number of work-up steps are removed; this has the advantage of reducing the plant ‘down time’ and higher through yields.

In a highly preferred embodiment, there is provided a process for the preparation of a compound of Formula 1(i):

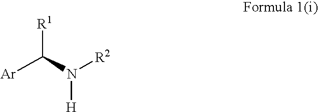

wherein

Ar represents an optionally substituted hydrocarbyl or an optionally substituted heterocyclyl group comprising an aromatic moiety; and

R¹ and R² each independently represent an optionally substituted hydrocarbyl or an optionally substituted heterocyclyl group;

said process comprising:
a) reducing a compound of Formula 2 with a stereoselective reduction system to form a compound of Formula 3(i):

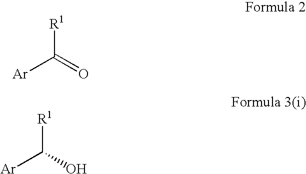

b) activating the compound of Formula 3(i) to form a compound of Formula 4(i):

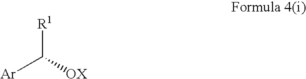

wherein X represents a leaving group; and
c) coupling the compound of Formula 4(i) to a compound of Formula 5:

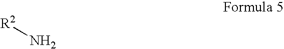

to form a compound of Formula 1(i).

Preferences for Ar, R¹, R², the compounds of Formula 2 and 5 and the stereoselective reduction system are as described herein before.

In a further preferred embodiment, the compound of Formula I(ii):

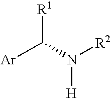

is prepared analogously from the corresponding compounds of Formula 3(ii) and 4(ii):

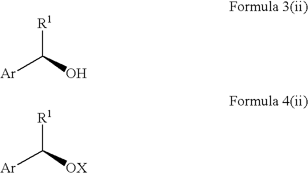

by reducing a compound of Formula 2 with a stereoselective reduction system to form a compound of Formula 3(ii); activating the compound of Formula 3(ii) to form a compound of Formula 4(ii); and coupling the compound of Formula 4(i) to a compound of Formula 5 to form a compound of Formula 1(ii).

Optionally, the compounds of Formula 1(i) and 1(ii) may be subjected to a further isolation step comprising diastereomeric crystallisation using, for example, a chiral acid such as malic acid, tartaric acid or camphorsulphonic acid.

Hydrocarbyl groups which may be represented by R¹ and R² independently include alkyl, alkenyl and aryl groups, and any combination thereof, such as aralkyl and alkaryl, for example benzyl groups.

Alkyl groups which may be represented by R¹ and R² include linear and branched alkyl groups comprising up to 20 carbon atoms, particularly from 1 to 7 carbon atoms and preferably from 1 to 5 carbon atoms. When the alkyl groups are branched, the groups often comprising up to 10 branch chain carbon atoms, preferably up to 4 branch chain atoms. In certain embodiments, the alkyl group may be cyclic, commonly comprising from 3 to 10 carbon atoms in the largest ring and optionally featuring one or more bridging rings. Examples of alkyl groups which may be represented by R¹ and R² include methyl, ethyl, propyl, 2-propyl, butyl, 2-butyl, t-butyl and cyclohexyl groups.

Alkenyl groups which may be represented by R¹ and R² include C₂₋₂₀, and preferably C₂₋₆ alkenyl groups. One or more carbon-carbon double bonds may be present. The alkenyl group may carry one or more substituents, particularly phenyl substituents. Examples of alkenyl groups include vinyl, styryl and indenyl groups.

Aryl groups which may be represented by R¹ and R² may contain 1 ring or 2 or more fused rings which may include cycloalkyl, aryl or heterocyclic rings. Examples of aryl groups which may be represented by R¹ and R² include phenyl, tolyl, fluorophenyl, chlorophenyl, bromophenyl, trifluoromethylphenyl, anisyl, naphthyl and ferrocenyl groups.

Perhalogenated hydrocarbyl groups which may be represented by R¹, R² and R³ independently include perhalogenated alkyl and aryl groups, and any combination thereof, such as aralkyl and alkaryl groups. Examples of perhalogenated alkyl groups which may be represented by R¹ and R² include —CF₃ and —C₂F₅.

Heterocyclic groups which may be represented by R¹ and R² independently include aromatic, saturated and partially unsaturated ring systems and may constitute 1 ring or 2 or more fused rings which may include cycloalkyl, aryl or heterocyclic rings. The heterocyclic group will contain at least one heterocyclic ring, the largest of which will commonly comprise from 3 to 7 ring atoms in which at least one atom is carbon and at least one atom is any of N, O, S or P. Examples of heterocyclic groups which may be represented by R¹ and R² include pyridyl, pyrimidyl, pyrrolyl, thiophenyl, furanyl, indolyl, quinolyl, isoquinolyl, imidazoyl and triazoyl groups.

When any of R¹ and R² is a substituted hydrocarbyl or heterocyclic group, the substituent(s) should be such so as not to adversely affect the rate or stereoselectivity of any of the reaction steps or the overall process. Optional substituents include halogen, cyano, nitro, hydroxy, amino, thiol, acyl, hydrocarbyl, heterocyclyl, hydrocarbyloxy, mono or di-hydrocarbylamino, hydrocarbylthio, esters, carbamates, carbonates, amides, sulphonyl and sulphonamido groups wherein the hydrocarbyl groups are as defined for R¹ above. One or more substituents may be present. Examples of R¹ and R² groups having more than one substituent present include —CF₃ and —C₂F₅.

Optionally substituted hydrocarbyl or an optionally substituted heterocyclyl group comprising an aromatic moiety which may be represented by Ar include optionally substituted aryl or heteroaryl groups, or an optionally substituted alkyl group, preferably a C₁₋₄ alkyl group, substituted by an optionally substituted aryl or heteroaryl group. Alkyl and aryl groups are as defined for R¹. Heteroaryl groups are heterocyclic groups as defined for R¹ which comprise at least one aromatic ring. Substituents include those substituents defined above for R¹. Substituents are commonly selected from the group consisting of optionally substituted alkoxy (preferably C₁₋₄-alkoxy), optionally substituted aryl (preferably phenyl), optionally substituted aryloxy (preferably phenoxy), polyalkylene oxide (preferably polyethylene oxide or polypropylene oxide), carboxy, phosphato, sulpho, nitro, cyano, halo, ureido, —SO₂F, hydroxy, ester, —NR^{a}R^{b}, —COR^{a}, —CONR^{a}R^{b}, —NHCOR^{a}, —OCONR^{a}R^{b}, carboxyester, sulphone, and —SO₂NR^{a}R^{b} wherein R^{a} and R^{b} are each independently H, optionally substituted aryl, especially phenyl, or optionally substituted alkyl (especially C₁₋₄-alkyl) or, in the case of —NR^{a}R^{b}, —CONR^{a}R^{b}, OCONR^{a}R^{b} and —SO₂NR^{a}R^{b}, R^{a} and R^{b} may also together with the nitrogen atom to which they are attached represent an aliphatic or aromatic ring system; or a combination thereof.

In many embodiments, R¹ is different from Ar, ie the compound of Formula 2 is prochiral. It is preferred that R¹ represents a C₁₋₄ alkyl group, and most preferably a methyl group.

In many especially preferred embodiments, the compound of Formula 2 is a compound of Formula 2a:

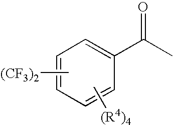

wherein R⁴ each independently represents hydrogen or a substituent group.

Preferable R⁴ are all hydrogen.

In certain preferred embodiments, the compound of Formula 2 is a compound of Formula 2b:

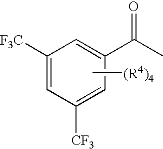

wherein R⁴ is as defined herein before.

Compounds of Formula 3 can be activated by employing methods known in the art for rendering a hydroxy group susceptible to displacement with an amino group. Examples of activation methods include the use of Mitsonubo conditions, phosphine and carbodiimide see for example Lawrence, Pharma Chem, (2002), 1(9), 12-14 and Hughes, Organic Reactions (New York) (1992), 42 335-656, the Mitsonubu conditions described in both being incorporated herein by reference.

In many embodiments, the compounds of Formula 3 are activated by reaction with a compound of formula X-L, wherein X is a leaving group precursor, and L is a halo group, especially a chloro or bromo group. Examples of preferred leaving group precursors which may be represented by X include acetyl, trifluoroacetyl, methanesulphonyl, trifluoromethylsulphonyl and toluenesulphonyl groups, and preferred compounds of formula X-L are the corresponding chloro compounds. In many other embodiments, the compounds of Formula 3 are activated by reaction with a compound of formula X—O—X, wherein X is as previously described. Examples of preferred leaving group precursors which may be represented by X include acetyl, trifluoroacetyl, methanesulphonyl, trifluoromethylsulphonyl and toluenesulphonyl groups. A highly preferred compound of formula X—O—X is methanesulphonic anhydride.

Preferably, the compounds of Formula 3a:

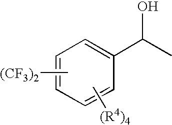

wherein R⁴ is as defined herein before, are activated by reaction with a compound of formula X-L, wherein X is as previously described. Examples of preferred leaving group precursors which may be represented by X include acetyl, trifluoroacetyl, methanesulphonyl, trifluoromethylsulphonyl and toluenesulphonyl groups. A highly preferred compound of formula X-L is methanesulphonyl chloride.

Most preferably, the compounds of Formula 3b:

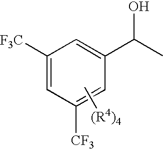

wherein R⁴ is as defined herein before, are activated by reaction with a compound of formula X-L, wherein X is an acetyl, trifluoroacetyl, methanesulphonyl, trifluoromethylsulphonyl or toluenesulphonyl group, to give a compound of Formula 4b which is reacted with a compound of Formula 5 to give a compound of Formula 1b.

Optionally, the compound of Formula 4b is isolated prior to reaction with the compound of Formula 5.

Preferably, for compounds of Formula 5, R² is an optionally substituted C₁₋₄-alkyl, optionally substituted phenyl or optionally substituted benzyl group. More preferably, R² is C₁₋₄-alkyl, phenyl or benzyl group. Most preferably, R² is a methyl group.

The reduction of compounds of Formula 2 is preferably accomplished employing a stereoselective reduction system. Stereoselective reduction systems include the use of chiral reducing agents, for example the use of metal hydrides with chiral complexes, the use of chiral coordinated transition metals in a catalysed transfer hydrogenation process, and the use of enzymatic reduction systems, for example whole cell or isolated enzyme based systems.

It is most preferred that the stereoselective reduction employs a chiral coordinated transition metal in a catalysed transfer hydrogenation process, and or the use of enzymatic reduction systems.

Enzymatic reduction systems include the use of enzymes in the form of whole cell systems or isolated enzymes. Thus the reduction of compounds of formula (2) to formula (3) in step (a) can be carried out using any enzyme suitable for reducing ketones to alcohols. Enzymes that are particularly suitable include oxidoreductases, reductases, and alcohol dehydrogenases. Microorganisms that can be used in the reduction process include: yeasts, bacteria, fungi, and plant and mammalian cells.

Examples of enzymes and microorganisms containing enzymes that may be deployed in the enzymatic reduction of compounds of formula (2) include enzymes and microorganisms described in M J Honman, Tetrahedron, 60, 789-797 (2004), geotrichum candidum BPCC 1118, WO 02/086126 and the oxidoreductase from Pichia Capsulata (WO 04/111083). The contents of each of these disclosures insofar as they relate to enzymes and microorganisms are specifically intended to be used in the reduction step of the process of the present invention and thus form part of the subject matter of the present invention. Whilst forming part of the subject matter of the present application the content of these disclosures are not reproduced here for reasons of brevity and because they are readily available.

In a preferred stereoselective reduction, a chiral coordinated transition metal catalysed transfer hydrogenation process is employed. Examples of such processes, and the catalysts, reagents and conditions employed therein include those disclosed in International patent application publication numbers WO97/20789, WO98/42643, and WO02/44111. The contents of each of these disclosures insofar as they relate to reaction conditions and catalysts are specifically intended to be used in the reduction step of the process of the present invention and thus form part of the subject matter of the present invention. Whilst forming part of the subject matter of the present application the content of these disclosures are not reproduced here for reasons of brevity and because they are readily available.

Preferred transfer hydrogenation catalysts for use in the process of the present invention have the general formula (a):

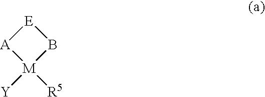

wherein:

R⁵ represents a neutral optionally substituted hydrocarbyl, a neutral optionally substituted perhalogenated hydrocarbyl, or an optionally substituted cyclopentadienyl ligand;

A represents an optionally substituted nitrogen;

B represents an optionally substituted nitrogen, oxygen, sulphur or phosphorous;

E represents a linking group;

M represents a metal capable of catalysing transfer hydrogenation; and

Y represents an anionic group, a basic ligand or a vacant site; and

provided that when Y is not a vacant site that at least one of A or B carries a hydrogen atom.

Preferably, at least one of A or B comprises a substituted nitrogen and the substituent has at least one chiral centre

Particularly preferred transfer hydrogenation catalysts are those Ru, Rh or Ir catalysts of the type described in WO97/20789, WO98/42643, and WO02/44111 which comprise an optionally substituted diamine ligand, for example an optionally substituted ethylene diamine ligand, wherein at least one nitrogen atom of the optionally substituted diamine ligand is substituted, preferably with a group containing a chiral centre, and a neutral aromatic ligand, for example p-cymene, or an optionally substituted cyclopentadiene ligand, for example pentamethylcyclopentadiene.

Highly preferred transfer hydrogenation catalysts for use in the process of the present invention are of general Formula (A):

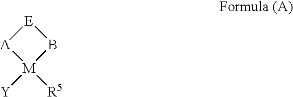

wherein:

R⁵ represents a neutral optionally substituted hydrocarbyl, a neutral optionally substituted perhalogenated hydrocarbyl, or an optionally substituted cyclopentadienyl ligand;

A represents —NR⁶—, —NR⁷—, —NHR⁶, —NR⁶R⁷ or —NR⁶R⁷ where R⁸ is H, C(O)R⁸, SO₂R⁸, C(O)NR⁸R¹², C(S)NR⁸R¹², C(═NR¹²)SR¹³ or C(═NR¹²)OR¹³, R⁷ and R⁸ each independently represents an optionally substituted hydrocarbyl, perhalogenated hydrocarbyl or an optionally substituted heterocyclyl group, and R¹² and R¹³ are each independently hydrogen or a group as defined for R⁸;

B represents —O—, —OH, OR⁹, —S—, —SH, SR⁹, —NR⁹—, —NR¹⁰—, —NHR¹⁰, —NR⁹R¹⁰, —NR⁹R¹¹, —PR⁹— or —PR⁹R¹¹ where R¹⁰ is H, C(O)R¹¹, SO₂R¹¹, C(O)NR¹¹R¹⁴, C(S)NR¹¹R¹⁴, C(═NR¹⁴)SR¹⁵ or C(═NR¹⁴)OR¹⁵, R⁹ and R¹¹ each independently represents an optionally substituted hydrocarbyl, perhalogenated hydrocarbyl or an optionally substituted heterocyclyl group, and R¹⁴ and R¹⁵ are each independently hydrogen or a group as defined for R¹¹;

E represents a linking group;

M represents a metal capable of catalysing transfer hydrogenation; and

Y represents an anionic group, a basic ligand or a vacant site; and

provided that when Y is not a vacant site that at least one of A or B carries a hydrogen atom.

Highly preferred are transfer hydrogenation catalysts of Formula (A) wherein at least one of A or B comprises a substituted nitrogen. When A or B comprises a substituted nitrogen, optionally the substituent has at least one chiral centre.

The catalytic species is believed to be substantially as represented in the above formula. It may be introduced on a solid support.

Optionally substituted hydrocarbyl groups represented by R⁷⁻⁹ or R¹¹⁻¹³ include alkyl, alkenyl, alkynyl and aryl groups, and any combination thereof, such as aralkyl and alkaryl, for example benzyl groups.

Alkyl groups which may be represented by R⁷⁻⁹ or R¹¹⁻¹³ include linear and branched alkyl groups comprising 1 to 20 carbon atoms, particularly from 1 to 7 carbon atoms and preferably from 1 to 5 carbon atoms. In certain embodiments, the alkyl group may be cyclic, commonly comprising from 3 to 10 carbon atoms in the largest ring and optionally featuring one or more bridging rings. Examples of alkyl groups which may be represented by R⁷⁻⁹ or R¹¹⁻¹³ include methyl, ethyl, propyl, 2-propyl, butyl, 2-butyl, t-butyl and cyclohexyl groups.

Alkenyl groups which may be represented by one or more of R⁷⁻⁹ or R¹¹⁻¹³ include C₂₋₂₀, and preferably C₂₋₆ alkenyl groups. One or more carbon-carbon double bonds may be present. The alkenyl group may carry one or more substituents, particularly phenyl substituents.

Alkynyl groups which may be represented by one or more of R⁷⁻⁹ or R¹¹⁻¹³ include C₂₋₂₀, and preferably C₂₋₁₀ alkynyl groups. One or more carbon-carbon triple bonds may be present. The alkynyl group may carry one or more substituents, particularly phenyl substituents. Examples of alkynyl groups include ethynyl, propyl and phenylethynyl groups.

Aryl groups which may be represented by one or more of R⁷⁻⁹ or R¹¹⁻¹³ may contain 1 ring or 2 or more fused or bridged rings which may include cycloalkyl, aryl or heterocyclic rings. Examples of aryl groups which may be represented by R⁷⁻⁹ or R¹¹⁻¹³ include phenyl, tolyl, fluorophenyl, chlorophenyl, bromophenyl, trifluoromethylphenyl, anisyl, naphthyl and ferrocenyl groups.

Perhalogenated hydrocarbyl groups which may be represented by one or more of R⁷⁻⁹ or R¹¹⁻¹³ independently include perhalogenated alkyl and aryl groups, and any combination thereof, such as aralkyl and alkaryl groups. Examples of perhalogenated alkyl groups which may be represented by R⁷⁻⁹ or R¹¹⁻¹³ include —CF₃ and —C₂F₅.

Heterocyclic groups which may be represented by one or more of R⁷⁻⁹ or R¹¹⁻¹³ independently include aromatic, saturated and partially unsaturated ring systems and may comprise 1 ring or 2 or more fused rings which may include cycloalkyl, aryl or heterocyclic rings. The heterocyclic group will contain at least one heterocyclic ring, the largest of which will commonly comprise from 3 to 7 ring atoms in which at least one atom is carbon and at least one atom is any of N, O, S or P. Examples of heterocyclic groups which may be represented by R⁷⁻⁹ or R¹¹⁻¹³ include pyridyl, pyrimidyl, pyrrolyl, thiophenyl, furanyl, indolyl, quinolyl, isoquinolyl, imidazolyl and triazolyl groups.

When any of R⁷⁻⁹ or R¹¹⁻¹³ is a substituted hydrocarbyl or heterocyclic group, the substituent(s) should be such so as not to adversely affect the rate or stereoselectivity of the reaction. Optional substituents include halogen, cyano, nitro, hydroxy, amino, imino, thiol, acyl, hydrocarbyl, perhalogenated hydrocarbyl, heterocyclyl, hydrocarbyloxy, mono or di-hydrocarbylamino, hydrocarbylthio, esters, carboxy, carbonates, amides, sulphonyl and sulphonamido groups wherein the hydrocarbyl groups are as defined for R⁷⁻⁹ or R¹¹⁻¹³ above. One or more substituents may be present. R⁷⁻⁹ or R¹¹⁻¹³ may each contain one or more chiral centres.

The neutral optionally substituted hydrocarbyl or perhalogenated hydrocarbyl ligand which may be represented by R⁵ includes optionally substituted aryl and alkenyl ligands.

Optionally substituted aryl ligands which may be represented by R⁵ may contain 1 ring or 2 or more fused rings which include cycloalkyl, aryl or heterocyclic rings. Preferably, the ligand comprises a 6 membered aromatic ring. The ring or rings of the aryl ligand are often substituted with hydrocarbyl groups. The substitution pattern and the number of substituents will vary and may be influenced by the number of rings present, but often from 1 to 6 hydrocarbyl substituent groups are present, preferably 2, 3 or 6 hydrocarbyl groups and more preferably 6 hydrocarbyl groups. Preferred hydrocarbyl substituents include methyl, ethyl, iso-propyl, menthyl, neomenthyl and phenyl. Particularly when the aryl ligand is a single ring, the ligand is preferably benzene or a substituted benzene. When the ligand is a perhalogenated hydrocarbyl, preferably it is a polyhalogenated benzene such as hexachlorobenzene or hexafluorobenzene. When the hydrocarbyl substitutents contain enantiomeric and/or diastereomeric centres, it is preferred that the enantiomerically and/or diastereomerically purified forms of these are used. Benzene, p-cymyl, mesitylene and hexamethylbenzene are especially preferred aryl ligands.

Optionally substituted alkenyl ligands which may be represented by R⁵ include C₂₋₃₀, and preferably C₆₋₁₂, alkenes or cycloalkenes with preferably two or more carbon-carbon double bonds, preferably only two carbon-carbon double bonds. The carbon-carbon double bonds may optionally be conjugated to other unsaturated systems which may be present, but are preferably conjugated to each other. The alkenes or cycloalkenes may be substituted preferably with hydrocarbyl substituents. When the alkene has only one double bond, the optionally substituted alkenyl ligand may comprise two separate alkenes. Preferred hydrocarbyl substituents include methyl, ethyl, iso-propyl and phenyl. Examples of optionally substituted alkenyl ligands include cycloocta-1,5-diene and 2,5-norbornadiene. Cyclo-octa-1,5-diene is an especially preferred alkenyl ligand.

Optionally substituted cyclopentadienyl groups which may be represented by R⁵ include cyclopentadienyl groups capable of eta-5 bonding. The cyclopentadienyl group is often substituted with from 1 to 5 hydrocarbyl groups, preferably with 3 to 5 hydrocarbyl groups and more preferably with 5 hydrocarbyl groups. Preferred hydrocarbyl substituents include methyl, ethyl and phenyl. When the hydrocarbyl substitutents contain enantiomeric and/or diastereomeric centres, it is preferred that the enantiomerically and/or diastereomerically purified forms of these are used. Examples of optionally substituted cyclopentadienyl groups include cyclopentadienyl, pentamethylcyclopentadienyl, pentaphenylcyclopentadienyl, tetraphenylcyclopentadienyl, ethyltetramethylpentadienyl, menthyltetraphenylcyclopentadienyl, neomenthyltetraphenylcyclopentadienyl, menthylcyclopentadienyl, neomenthylcyclopentadienyl, tetrahydroindenyl, menthyltetrahydroindenyl and neomenthyltetrahydroindenyl groups. Pentamethylcyclopentadienyl is an especially preferred cyclopentadienyl ligand.

When either A or B is an amide group represented by —NR⁸—, —NHR⁶, NR⁶R⁷, —NR¹⁰—, —NHR¹⁰ or NR⁹R¹⁰ wherein R⁷ and R⁹ are as hereinbefore defined, and where R⁶ or R¹⁰ is an acyl group represented by —C(O)R⁸ or —C(O)R¹¹, R⁸ and R¹¹ independently are often linear or branched C₁₋₇alkyl, C₁₋₈-cycloalkyl or aryl, for example phenyl. Examples of acyl groups which may be represented by R⁶ or R¹⁰ include benzoyl, acetyl and halogenoacetyl, especially trifluoroacetyl groups.

When either A or B is present as a sulphonamide group represented by —NR⁶—, —NHR⁶, NR⁶R⁷, —NR¹⁰—, —NHR¹⁰ or NR⁹R¹⁰ wherein R⁷ and R⁹ are as hereinbefore defined, and where R⁶ or R¹⁰ is a sulphonyl group represented by —S(O)₂R⁸ or —S(O)₂R¹¹, R⁸ and R¹¹ independently are often linear or branched C₁₋₁₂alkyl, C₁₋₁₂cycloalkyl or aryl, for example phenyl. Preferred sulphonyl groups include methanesulphonyl, trifluoromethanesulphonyl, more preferably p-toluenesulphonyl groups, naphthylsulphonyl groups and camphorsulphonyl.

When either of A or B is present as a group represented by —NR⁶—, —NHR⁶, NR⁶R⁷, —NR¹⁰—, —NHR¹⁰ or NR⁹R¹⁰ wherein R⁷ and R⁹ are as hereinbefore defined, and where R⁶ or R¹⁰ is a group represented by C(O)NR⁸R¹², C(S)NR⁸R¹², C(═NR¹²)SR¹³, C(═NR¹²)OR¹³, C(O)NR¹¹R¹⁴, C(S)NR¹¹R¹⁴, C(═NR⁴)SR¹⁵ or C(═NR¹⁴)OR¹⁵, R⁸ and R¹¹ independently are often linear or branched C₁₋₈alkyl, such as methyl, ethyl, isopropyl, C₁₋₈cycloalkyl or aryl, for example phenyl, groups and R¹²⁻¹⁵ are often each independently hydrogen or linear or branched C₁₋₈alkyl, such as methyl, ethyl, isopropyl, C₁₋₈cycloalkyl or aryl, for example phenyl, groups.

When B is present as a group represented by —OR⁹, —SR⁹, —PR⁹— or —PR⁹R¹¹, R⁹ and R¹¹ independently are often linear or branched C₁₋₄alkyl, such as methyl, ethyl, isopropyl, C₁₋₈cycloalkyl or aryl, for example phenyl.

It will be recognised that the precise nature of A and B will be determined by whether A and/or B are formally bonded to the metal or are coordinated to the metal via a lone pair of electrons.

The groups A and B are connected by a linking group E. The linking group E achieves a suitable conformation of A and B so as to allow both A and B to bond or coordinate to the metal, M. A and B are commonly linked through 2, 3 or 4 atoms. The atoms in E linking A and B may carry one or more substituents. The atoms in E, especially the atoms alpha to A or B, may be linked to A and B, in such a way as to form a heterocyclic ring, preferably a saturated ring, and particularly a 5, 6 or 7-membered ring. Such a ring may be fused to one or more other rings. Often the atoms linking A and B will be carbon atoms. Preferably, one or more of the carbon atoms linking A and B will carry substituents in addition to A or B. Substituent groups include those which may substitute R⁷⁻⁹ or R¹¹⁻¹³ as defined above. Advantageously, any such substituent groups are selected to be groups which do not coordinate with the metal, M. Preferred substituents include halogen, cyano, nitro, sulphonyl, hydrocarbyl, perhalogenated hydrocarbyl and heterocyclyl groups as defined above. Most preferred substituents are C₁₋₈ alkyl groups, and phenyl groups. Most preferably, A and B are linked by two carbon atoms, and especially an optionally substituted ethyl moiety. When A and B are linked by two carbon atoms, the two carbon atoms linking A and B may comprise part of an aromatic or aliphatic cyclic group, particularly a 5, 6 or 7-membered ring. Such a ring may be fused to one or more other such rings. Particularly preferred are embodiments in which E represents a 2 carbon atom separation and one or both of the carbon atoms carries an optionally substituted aryl group as defined above or E represents a 2 carbon atom separation which comprises a cyclopentane or cyclohexane ring, optionally fused to a phenyl ring.

E preferably comprises part of a compound having at least one stereospecific centre. Where any or all of the 2, 3 or 4 atoms linking A and B are substituted so as to define at least one stereospecific centre on one or more of these atoms, it is preferred that at least one of the stereospecific centres be located at the atom adjacent to either group A or B. When at least one such stereospecific centre is present, it is advantageously present in an enantiomerically purified state.

When B represents —O— or —OH, and the adjacent atom in E is carbon, it is preferred that B does not form part of a carboxylic group.

Compounds which may be represented by A-E-B, or from which A-E-B may be derived by deprotonation, are often aminoalcohols, including 4-aminoalkan-1-ols, 1-aminoalkan-4-ols, 3-aminoalkan-1-ols, 1-aminoalkan-3-ols, and especially 2-aminoalkan-1-ols, 1-aminoalkan-2-ols, 3-aminoalkan-2-ols and 2-aminoalkan-3-ols, and particularly 2-aminoethanols or 3-aminopropanols, or are diamines, including 1,4-diaminoalkanes, 1,3-diaminoalkanes, especially 1,2- or 2,3-diaminoalkanes and particularly ethylenediamines. Further aminoalcohols that may be represented by A-E-B are 2-aminocyclopentanols and 2-aminocyclohexanols, preferably fused to a phenyl ring. Further diamines that may be represented by A-E-B are 1,2-diaminocyclopentanes and 1,2-diaminocyclohexanes, preferably fused to a phenyl ring. The amino groups may advantageously be N-tosylated. When a diamine is represented by A-E-B, preferably at least one amino group is N-tosylated. The aminoalcohols or diamines are advantageously substituted, especially on the linking group, E, by at least one alkyl group, such as a C₁₋₄-alkyl, and particularly a methyl, group or at least one aryl group, particularly a phenyl group.

Specific examples of compounds which can be represented by A-E-B and the protonated equivalents from which they may be derived are:

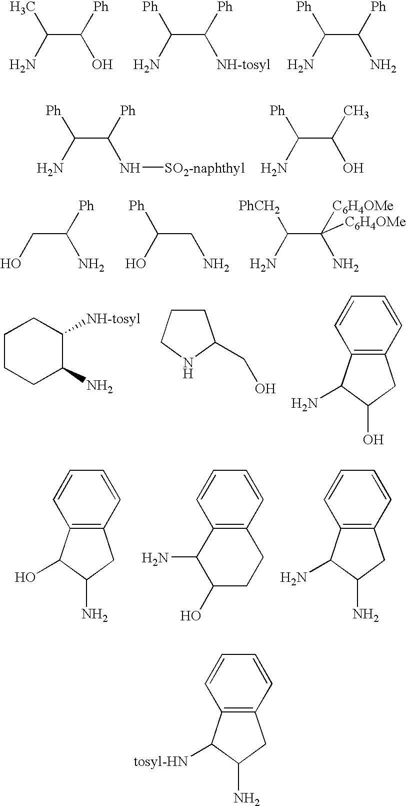

Preferably, the enantiomerically and/or diastereomerically purified forms of these are used. Examples include (1S,2R)-(+)-norephedrine, (1R,2S)-(+)-cis-1-amino-2-indanol, (1S,2R)-2-amino-1,2-diphenylethanol, (1S,2R)-(−)cis-1-amino-2-indanol, (1R,2S)-(−)-norephedrine, (S)-(+)-2-amino-1-phenylethanol, (1R,2S)-2-amino-1,2-diphenylethanol, N-tosyl-(1R,2R)-1,2-diphenylethylenediamine, N-tosyl-(1S,2S)-1,2-diphenylethylenediamine, (1R,2S)-cis-1,2-indandiamine, (1S,2R)-cis-1,2-indandiamine, (R)-(−)-2-pyrrolidinemethanol and (S)-(+)-2-pyrrolidinemethanol.

Metals which may be represented by M include metals which are capable of catalysing transfer hydrogenation. Preferred metals include transition metals, more preferably the metals in Group VIII of the Periodic Table, especially ruthenium, rhodium or iridium. When the metal is ruthenium it is preferably present in valence state II. When the metal is rhodium or iridium it is preferably present in valence state I when R⁵ is a neutral optionally substituted hydrocarbyl or a neutral optionally substituted perhalogenated hydrocarbyl ligand, and preferably present in valence state III when R⁵ is an optionally substituted cyclopentadienyl ligand.

It is preferred that M, the metal, is rhodium present in valence state III and R⁵ is an optionally substituted cyclopentadienyl ligand.

Anionic groups which may be represented by Y include hydride, hydroxy, hydrocarbyloxy, hydrocarbylamino and halogen groups. Preferably when a halogen is represented by Y, the halogen is chloride. When a hydrocarbyloxy or hydrocarbylamino group is represented by Y, the group may be derived from the deprotonation of the hydrogen donor utilised in the reaction.

Basic ligands which may be represented by Y include water, C₁₋₄ alcohols, C₁₋₈ primary or secondary amines, or the hydrogen donor which is present in the reaction system. A preferred basic ligand represented by Y is water.

Most preferably, A-E-B, R⁵ and Y are chosen so that the catalyst is chiral. When such is the case, an enantiomerically and/or diastereomerically purified form is preferably employed. Such catalysts are most advantageously employed in asymmetric transfer hydrogenation processes. In many embodiments, the chirality of the catalyst is derived from the nature of A-E-B.

Preferred catalysts are of Formula B(i-ii) and C(i-iv):

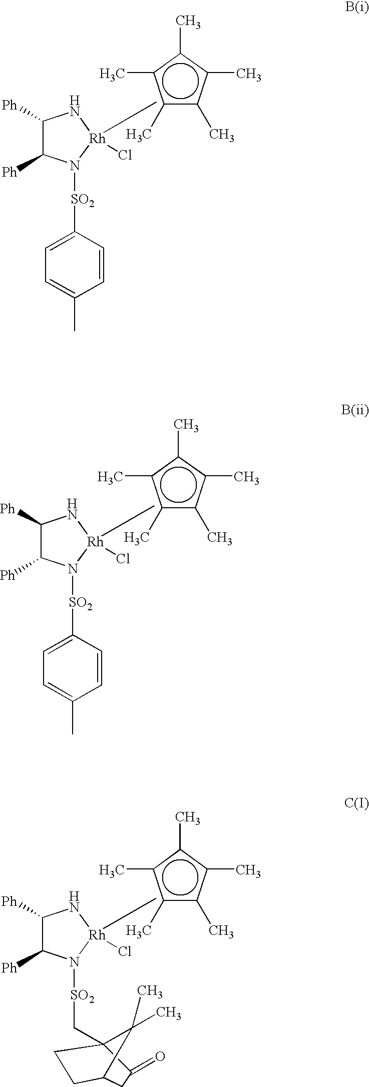

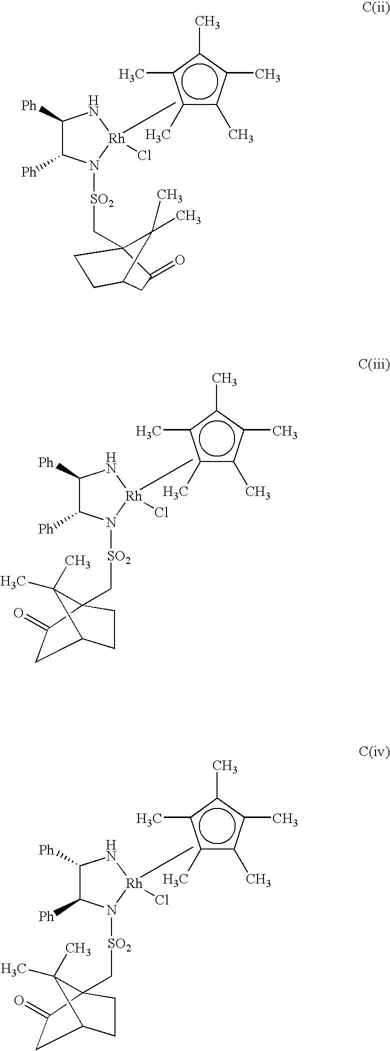

Catalysts of Formula B(i) and B(ii) are most preferred.

The preferred catalyst may be prepared in-situ preferably by combining a chiral bidentate nitrogen ligand with a Rh(III) metal complex containing a substituted cyclopentadienyl ligand. Preferably a solvent is present in this operation. The solvent used may be any solvent which does not adversely effect the formation of the catalyst. These solvents include acetonitrile, ethylacetate, toluene, methanol, tetrahydrofuran, ethylmethyl ketone, dimethyl formamide and mixtures thereof. Preferably the solvent is THF or dimethyl formamide.

Any suitable reductant may be used in the preferred embodiment of step (a), examples of reductants able to be used in this process include hydrogen donors including hydrogen, primary and secondary alcohols, primary and secondary amines, carboxylic acids and their esters and salts, readily dehydrogenatable hydrocarbons, clean reducing agents, and any combination thereof.

Primary and secondary alcohols which may be employed in the preferred embodiment of step (a) as hydrogen donors comprise commonly from 1 to 10 carbon atoms, preferably from 2 to 7 carbon atoms, and more preferably 3 or 4 carbon atoms. Examples of primary and secondary alcohols which may be represented as hydrogen donors include methanol, ethanol, propan-1-ol, propan-2-ol, butan-1-ol, butan-2-ol, cyclopentanol, cyclohexanol, benzylalcohol, and menthol, especially propan-2-ol and butan-2-ol.

Primary and secondary amines which may be employed in the preferred embodiment of step (a) as hydrogen donors comprise commonly from 1 to 20 carbon atoms, preferably from 2 to 14 carbon atoms, and more preferably 3 or 8 carbon atoms. Examples of primary and secondary amines which may act as hydrogen donors include ethylamine, propylamine, isopropylamine, butylamine, isobutylamine, hexylamine, diethylamine, dipropylamine, di-isopropylamine, dibutylamine, di-isobutylamine, dihexylamine, benzylamine, dibenzylamine and piperidine. When the hydrogen donor is an amine, primary amines are preferred, especially primary amines comprising a secondary alkyl group, particularly isopropylamine and isobutylamine.

Carboxylic acids and their esters which in a preferred embodiment of step (a) may act as hydrogen donors comprise commonly from 1 to 10 carbon atoms, preferably from 1 to 3 carbon atoms. In certain embodiments, the carboxylic acid is advantageously a beta-hydroxy-carboxylic acid. Esters may be derived from the carboxylic acid and a C₁₋₁₀ alcohol. Examples of carboxylic acids which may be employed as hydrogen donors include formic acid, lactic acid, ascorbic acid and mandelic acid, especially formic acid.

In certain preferred embodiments, when a carboxylic acid is employed as hydrogen donor, at least some of the carboxylic acid is preferably present as salt, preferably an amine, ammonium or metal salt. Preferably, when a metal salt is present the metal is selected from the alkali or alkaline earth metals of the periodic table, and more preferably is selected from the group I elements, such as lithium, sodium or potassium. Amines which may be used to form such salts include; primary, secondary and tertiary amines which comprise from 1 to 20 carbon atoms. Cyclic amines, both aromatic and non-aromatic, may also be used. Tertiary amines, especially trialkylamines, are preferred. Examples of amines which may be used to form salts include; trimethylamine, triethylamine, di-isopropylethylamine and pyridine. The most preferred amine is triethylamine.

When at least some of the carboxylic acid is present as an amine salt, particularly when a mixture of formic acid and triethylamine is employed, the mole ratio of acid to amine is between 1:1 and 50:1 and preferably between 1:1 and 10:1, and most preferably about 5:2. When at least some of the carboxylic acid is present as a metal salt, particularly when a mixture of formic acid and a group I metal salt is employed, the mole ratio of acid to metal ions present is between 1:1 and 50:1 and preferably between 1:1 and 10:1, and most preferably about 2:1. The ratios of acid to salts may be maintained during the course of the reaction by the addition of either component, but usually by the addition of the carboxylic acid.

Readily dehydrogenatable hydrocarbons which may be employed in step (a) as hydrogen donors comprise hydrocarbons which have a propensity to aromatise or hydrocarbons which have a propensity to form highly conjugated systems. Examples of readily dehydrogenatable hydrocarbons which may be employed by as hydrogen donors include cyclohexadiene, cyclohexene, tetralin, dihydrofuran and terpenes.

Clean reducing agents able to act as hydrogen donors comprise reducing agents with a high reduction potential, particularly those having a reduction potential relative to the standard hydrogen electrode of greater than about −0.1 eV, often greater than about −0.5 eV, and preferably greater than about −1 eV. Examples of suitable clean reducing agents include hydrazine and hydroxylamine.

Preferred hydrogen donors in the preferred embodiment of step (a) are propan-2-ol, butan-2-ol, triethylammonium formate and a mixture of triethylammonium formate and formic acid.

The most preferred transfer hydrogenation processes employ triethylamine-formic acid as hydrogen source.

When the hydrogen donor is a primary or secondary alcohol, the process is carried out preferably in the presence of a base, especially when Y is not a vacant site. The pKₐ of the base is preferably at least 8.0, especially at least 10.0. Convenient bases are the hydroxides, alkoxides and carbonates of alkali metals; tertiary amines and quaternary ammonium compounds. Preferred bases are sodium 2-propoxide and triethylamine. The quantity of base used can be up to 5.0, commonly up to 3.0, often up to 2.5 and especially in the range 1.0 to 3.5, by moles of the catalyst.

Although gaseous hydrogen may be present, the process is normally operated in the absence of gaseous hydrogen since it appears to be unnecessary.

Preferably, the reaction is often carried out under an inert atmosphere, for example nitrogen. More preferably, the reaction is sparged with inert gas.

When the product(s) from dehydrogenation of the hydrogen donor is volatile, for example boils at under 100° C., the removal of this volatile product is preferred. The removal can be accomplished by the use of inert gas sparging. More preferably, the removal is accomplished by distillation preferably at less than atmospheric pressure. When reduced pressure distillation is employed, the pressure is often no more than 500 mmHg, commonly no more than 200 mmHg, preferably in the range of from 5 to 100 mmHg, and most preferably from 10 to 80 mmHg.

Suitably the process is carried out at temperatures in the range of from −78 to 150° C., preferably from −20 to 110° C. and more preferably from −5 to plus 60° C. The initial concentration of the substrate, a compound of formula (2), is suitably in the range 0.05 to 1.0 and, for convenient larger scale operation, can be for example up to 6.0 more especially 0.75 to 2.0, on a molar basis. The molar ratio of the substrate to catalyst is suitably no less than 50:1 and can be up to 50000:1, preferably between 250:1 and 5000:1 and more preferably between 500:1 and 2500:1. The hydrogen donor is preferably employed in a molar excess over the substrate, especially up to 5 fold, and often up to 20 fold. When the hydrogen donor is a primary or secondary alcohol and the alcohol is used as a solvent, the molar excess may be even greater, for example up to 500 fold. Reaction times are typically in the range of from 1.0 min to 24 h, especially up to 8 h and conveniently about 3-6 h. It appears that substantially shorter times than those disclosed in the above-mentioned publications are made practicable by the invention. After reaction, the mixture is worked up by standard procedures. A reaction solvent may be present, for example acetonitrile, toluene, methyl t-butyl ether, alcohols, halogenated hydrocarbons or, conveniently, the hydrogen donor when the hydrogen donor is liquid at the reaction temperature, particularly when the hydrogen donor is a primary or secondary alcohol or a primary or secondary amine. Although it is possible to operate in the substantial absence of water, the use of water and an organic solvent to operate the process as a two phase system is preferred. Such two phase systems may ameliorate the production of hydrogen.

According to a second aspect of the present invention there is provided a process for the transfer hydrogenation of a compound of formula (6) to produce a compound of formula (7)

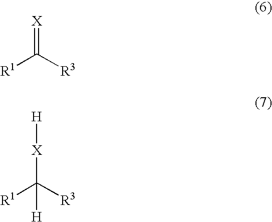

wherein:
X represents O; and
R¹ and R³ each independently represents a hydrogen atom, an optionally substituted hydrocarbyl, a perhalogenated hydrocarbyl or an optionally substituted heterocyclyl group, or R¹ & R³ optionally being linked in such a way as to form an optionally substituted ring(s),
   said process comprising reacting the compound of formula (6) with a hydrogen donor in the presence of a transfer hydrogenation catalyst in a multi-phase system.

Optionally substituted hydrocarbyl groups, perhalogenated hydrocarbyl groups and optionally substituted heterocyclyl groups which may be represented by R³ are as defined for R¹ above. It is preferred that R¹ and R³ are different.

The multi-phase system preferably comprises two or more liquid phases. More preferably the multi-phase system is a two phase system comprising a water immiscible solvent phase and an aqueous or water phase.

When a two phase system comprising a water immiscible solvent phase and an aqueous or water phase is employed, the water immiscible solvent phase may be dispersed in the continuous aqueous or water phase or the aqueous or water phase may be dispersed in the continuous water immiscible solvent phase.

Preferably, the transfer hydrogenation catalyst is soluble in the water immiscible solvent phase. Preferably the hydrogen donor is soluble in the aqueous or water phase.

Preferred transfer hydrogenation catalysts are those transfer hydrogenation catalysts described herein before above which are soluble in water immiscible solvents.

Preferred transfer hydrogenation catalysts which are soluble in water immiscible solvents are those optionally substituted transfer hydrogenation catalysts which do not comprise substitutents that confer water solubility. For example, substitutents that confer water solubility include sulphonic acid groups or salts thereof.

Preferably, the liquid water immiscible phase comprises the compound of formula (6) and optionally one or more immiscible solvents. Preferred water immiscible solvents include those polar and non-polar organic solvents described herein before above which are partially or fully water immiscible. Preferred water immiscible solvents include t-butyl acetate, THF. Dichloromethane is a highly preferred water immiscible solvent.

In a highly preferred embodiment, when the compound of formula (6) is a liquid at the temperature at which the process is operated and the compound of formula (6) is water immiscible or has only partial water solubility, no water immiscible solvent is employed. The compound of formula (6) may be present as a neat oil in a preferred embodiment.

Optionally a phase transfer catalyst may be present. Surprisingly it has been found that the use of phase transfer catalysts may increase reaction rates. Examples of phase transfer catalysts include quaternary ammonium salts such as halides and sulphates, for example (Bu)₄N⁺SO₄⁻. The use of phase transfer catalysts is preferred.

The invention is illustrated by the following Examples.

### EXPERIMENTAL

### Experiment 1

### Stage 1

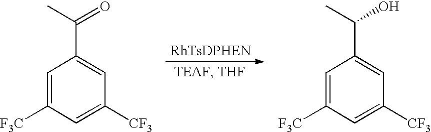

### Materials

| | | | | | M.W. |
|---|---|---|---|---|---|
| Name | Mols | Amount | | Equiv | (g/mol) |
| 3,5- | 0.234 | 60 | g | 1 | 256.15 |
| bis(trifluoromethyl)- | | | | | |
| acetophenone | | | | | |
| HCOOH* | 1.03 | 39.2 | ml | 4.4 | 46.03 |
| Et₃N* | 0.41 | 57.2 | ml | 1.76 | 101.19 |
| THF | — | 117 | ml | — | 72.11 |
| [RhCp*Cl₂]₂ | 0.585 mmol | 0.36 | g | 1/400 | 618 |
| S,S-TsDPEN | 1.17 mmol | 0.428 | g | 1/200 | 366 |
| toluene | — | 100 | ml | — | 84.93 |
| NaOH (2M) | — | 200 | ml | — | 40 |

*Charged as a triethylamine/formic acid mixture = TEAF = 90 mL of the mixture

### Method

[RhCp*Cl₂]₂ and S,S-TsDPEN were charged to a split necked flask and the vessel placed under a nitrogen atmosphere. THF was charged to the vessel at ambient temperature with stirring and a nitrogen purge. To this was charged 3,5-bis(trifluoromethyl)acetophenone and the contents stirred for 15 mins. The TEAF (triethylamine/formic acid mixture) was then charged dropwise over 30 mins. The reaction was allowed to stir at 20° C. and the reaction monitored by GC (complete after approx 1 hour).

The reaction was quenched by charging NaOH (2M) ensuring that the reaction temperature does not exceed 30° C.

The solution was stirred vigorously for 30 mins and allowed to settle for 30 mins. The lower organic layer was run off and fresh toluene charged to the separating vessel. The solution was stirred vigorously for 30 mins and allowed to settle for 30 mins and the lower organic layer was run off. The organic layers were combined and concentrated to ⅓ volume. This solution was used directly in the next stage. (Yield: >98%, 82% ee)

### Stage 2

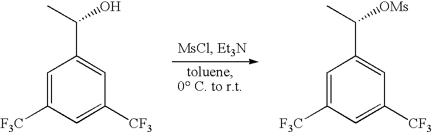

### Materials

| | | | | M.W. |
|---|---|---|---|---|
| Name | Mols | amount | Equiv. | (g/mol) |
| 3,5- | 0.232 | 60 g | 1 | 258.15 |
| bis(trifluoromethyl)- | | | | |
| phenylethanol | | | | |
| methanesulophonyl | 0.244 | 19.3 ml | 1.05 | 114.55 |
| choride | | | | |
| Et₃N | 0.349 | 48.7 ml | 1.5 | 101.19 |
| toluene | — | 500 ml | — | 84.93 |
| water | — | 600 ml | — | 18 |

### Method

The stage 1 toluene solution, triethylamine and toluene were charged to a nitrogen filled split neck flask and cooled to 5° C. with stirring. The methane sulphonyl chloride was charged dropwise ensuring that the reaction temperature does not exceed 15° C. The reaction mass was warmed to 20° C. over 1 hour. Water was cautiously charged keeping the temp below 30° C. The organic layer was washed twice with water. The toluene layer was used directly in the next stage. (Yield: >98%; 82% ee)

### Stage 3

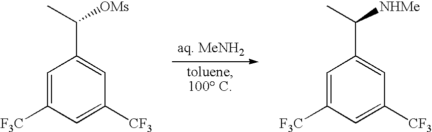

### Materials

| | | | | M.W. |
|---|---|---|---|---|
| Name | Mols | amount | Equiv. | (g/mol) |
| 3,5-bis(trifluoromethyl)- | 0.234 | 78.7 g | 1 | 336 |
| benzylmesylate | | | | |
| 40% aqueous | 0.585 | 237 ml | 2.5 | 31 |
| methylamine | | | | |

### Method

The stage 2 toluene solution and the aqueous methylamine (40 wt % solution) was charged to a Parr reactor. The vessel was sealed and warmed to 50° C. (1.8 bar max). The reaction was completed after 48 hrs. The two layers were transferred to a separating funnel and separated. The organic layer was washed twice with water and once with brine (⅓ volume each). (Yield: >98%; 79% ee)

### Stage 4

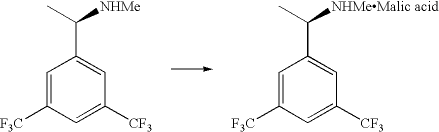

### Materials

| | | | | M.W. |
|---|---|---|---|---|
| Name | Mols | amount | Equiv. | (g/mol) |
| 3,5- | 0.11 | 31.7 g | 1 | 271.15 |
| bis(trifluoromethyl)- | | (250 ml of the | | |
| benzylmethylamine | | previous | | |
| | | solution) | | |
| L-malic acid | 0.11 | 14.83 | 1 | 134.09 |
| 2- propanol | — | 250 ml | — | — |
| ethyl acetate | — | 250 ml | — | — |

### Method

L-malic acid and 2-propanol were charged to a split necked flask and the flask placed under a nitrogen blanket. The mixture was heated to 60° C. until complete dissolution is observed when the vessel is cooled to 40° C. Stage 3 toluene solution is charged and the mixture distilled to ½ volume (some solids are formed during this distillation). Ethyl acetate is then charged and the mixture heated to 75° C. and held for 30 mins. The resulting solution is then cooled to 4° C. over 4 hours and held for 4 hours. The white/yellow crystals were collected by filtration and washed twice with cold ethyl acetate to afford the desired product as colourless crystals. Further crops of product can be obtained by concentrating the filtrates to ⅓ volume and allowing to cool to 0° C. The product is dried overnight in a vacuum oven at 40° C. (Yield: 33.2 g of the malic acid salt was obtained; 99% ee)

Overall yield (over 4 Stages)=69-80%

### Experiment 2

### Biphasic Reduction of 3,5-(bistrifluoromethyl)phenylacetophenone

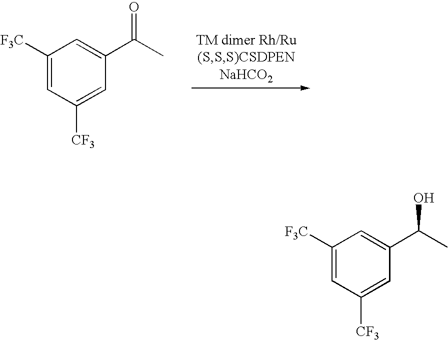

A reaction flask was flushed through with nitrogen and charged with a solution of sodium formate (33.1 g, 0.486 mol, 5 eq) in distilled water (131.4 g, 7.3 mol, 75 eq). Rh or Ru metal-dimer (0.39 mmol, 0.004 eq of Rh₂(Cp⁺)₂Cl₄ or Ru₂(p-cymyl)₂Cl₄) was added, followed by (S,S,S)CsDPEN ligand (0.332 g, 0.77 mmol, 0.008 eq) and the aqueous mixture agitated under nitrogen for 20 minutes.

An organic phase consisting of (3,5-bistrifluoromethyl)acetophenone (24.94 g, 97.4 mmol, 1 eq) and biphenyl (0.3 g, 1.93 mmol, 0.02 eq) in DCM (42.18 g, 0.497 mol, 5.1 eq) was made up giving a total organic volume of 49.4 ml. This organic solution was added to the aqueous whilst under agitation to form a well mixed, biphasic-aqueous continuous phase system, and the reaction allowed to proceed with GC sampling at regular intervals. Upon organic addition the solution changed from a pale orange to a red colour and any solids with low aqueous solubility dissolved. As reaction preceded the mixture slowly changed from a red to a dark brown colour, pH rose non-lineally from 7.0 to level out at 8.5. Reaction times were found to be 45 minutes for reaction using Rh-dimer and 700 minutes Ru-dimer. Reactions were worked up; agitation was ceased and the phases separated. The aqueous phase was washed with DCM (2×10 ml) and the organic phases combined and washed with distilled water (2×10 ml) followed by drying over anhydrous sodium sulphate and filtration. Next the dark brown solution was slurried with silica for 1 hour until the solution was clear, then the silica was filtered off and the solution concentrated in vacuo, to yield (3,5-Bistrifluoromethyl)phenylethanol as a white crystalline solid (17.15 g, 66.4 mmol, 68%). Enantiomeric excess when using Rh-dimer was found to be 83.0% and with Ru-dimer 81.5%.

(Note: biphenyl is present as an internal reference standard to assist in quantifying the GC results.)

### Experiment 3

Experiment 2 was repeated except that the ligand was pre-dissolved. The reaction was carried out as above but Rh-dimer was added to aqueous formate solution and agitated for 5 minutes, then CsDPEN ligand was added pre-dissolved in DCM (10.0 g, 118 mmol, 1.2 eq) and the mixture agitated for a further 15 minutes. Then solution of ketone/standard in DCM (32.18 g, 379 mmol, 3.9 eq) was added and the reaction monitored. Results show that while the rate of hydrogenation is similar, there is increased conversion (conversion was increased from 90.7% to 98.5%).

### Experiment 4

Experiment 2 was repeated using a phase transfer catalyst addition. The reaction was set up and allowed to run as before using the Ru-catalyst. After 60 minutes (Bu₄N)₂SO₄ PTC (5.66 g of a 50% wt solution in water, 0.05 eq) was added. The reaction rate increased instantaneously. The increase was approx 520%.

### Experiment 5

Reduction of 3,5-bis(trifluoromethyl)acetophenone using Ir (S,S) TSDPEN in aqueous formate solution

| | | | | MW |
|---|---|---|---|---|
| Material | Moles | Amount (g) | Equiv. | (g/mol) |
| 3,5- | 0.0975 | 25 | 1 | 256.15 |
| bis(trifluoromethyl) | | | | |
| acetophenone | | | | |
| Sodium formate | 0.493 | 33.5 | 5 | 68.01 |
| Water | 7.33 | 132 | 75 | 18 |
| Ir₂Cp*₂Cl₂ | 1.95 × 10⁻⁴ | 0.155 | 0.002 | 796.67 |
| (S,S) TSDPEN | 3.9 × 10⁻⁴ | 0.286 | 0.004 | 366.49 |
| Biphenyl | 1.62 × 10⁻³ | 0.25 | 0.017 | 154.21 |

Sodium formate was added to the water to make a 3.7M solution, which was then cooled to 10° C. The Ir dimer was added and stirred for 20 minutes. The (S,S) TSDPEN was then added to the mixture and allowed to stir for 10 minutes prior to the addition of the ketone. The reaction was complete after 24 hr. The suspension of product in aqueous solution was extracted twice with dichloromethane (2×40 ml). The organic layers were combined and passed through a silica plug twice to remove the catalyst before being reduced in vacuo to yield the white, crystalline product (17.3 g, 69% yield). The combined organic layers can be used directly in the next stage without the need to isolate the product. Analysis by chiral GC (Chiralsil-Dex, 25 m, 0.25 i.d., 0.25 mm film) showed the product to be 90.2% e.e (R).

### Experiment 6

Geotrichum candidum BPCC 1118 was grown aerobically in shake flasks containing a mineral salts medium pH 7.2, supplemented with glucose (5 g/litre), yeast extract (2 g/litre) and 2-propanol (15 g/litre). Cultures were incubated on a shaker at 28 degrees centigrade for 24 hours and the cells recovered by centrifugation. The recovered cell pellet was dehydrated by resuspension in 10 volumes of acetone, the cells were recovered by filtration and washed twice more with acetone before drying under vacuum to provide a free-flowing powder. Reactions were analysed by GC on a DB17 column (30 m×0.32 mm), using a temperature gradient (initial temperature 80 degrees C. held for 2.5 minutes, rising at 20 degrees per minute to 200 degrees) the starting material eluted at 3.8 minutes and the reduction product at 5.2 minutes. Chiral analysis was carried out by GC using a Chiraldex CB column (25 m×0.32 mm) on a temperature gradient (initial temperature 80 degrees held for 5 minutes, rising at 10 degrees per minute to a final temperature of 180 degrees and held for 2 minutes), the (S)-enantiomer eluted at 12.3 minutes and the (R)-enantiomer at 12.7 minutes. The reduction of 3,5-bis-(trifluoromethyl)-acetophenone (20 mg) was carried out in 2 ml of sodium phosphate buffer (pH7.5) containing acetone dried Geotrichum candidum cells (100 mg), nicotinamide adenine dinucleotide (1.5 mg) and 2-propanol (2.6 mg) incubated at 28 degrees centigrade for 24 hours, the reaction conversion was 65% and the enantiomeric excess was >99% (S).

### Experiment 7

R-[3,5-bis(trifluoromethyl)phenyl]ethan-1-ol (99.9% EE) in toluene was charged to the vessel and the vessel placed under a nitrogen blanket. This material can be obtained without isolation as a solution in toluene from Example 5 and Example 6 for example. Alternatively, the solid may be dissolved in toluene as required.

| | | | | M.W. |
|---|---|---|---|---|
| Material | Moles | Amount/g | Mol Ratio | g · mol |
| R-[3,5-bis(trifluoro- | 0.0332 | 22.0 | 1.00 | 258.15 |
| methyl)phenyl]ethan-1-ol | | | | |
| in toluene (approx 40% | | | | |
| strength) | | | | |
| Triethylamine | 0.0492 | 5.0 | 1.48 | 101.20 |
| Toluene line wash (1) | 0.0143 | 1.3 | 0.43 | 92.10 |
| Mesylchloride | 0.0359 | 4.1 | 1.08 | 114.55 |
| Toluene line wash (2) | 0.0273 | 2.5 | 0.82 | 92.10 |
| 10% HCl solution | 0.0369 | 13.5 | 1.11 | 36.50 |
| Water * 2 | 0.7857 | 14.2 | 23.64 | 18.02 |
| 40% Aqueous Methylamine | 0.1653 | 12.8 | 4.98 | 31.00 |
| Water * 3 | 0.6384 | 11.5 | 19.23 | 18.02 |
| 0.5M HCl * 2 | 0.0664 | 90.0 | 2.00 | 36.46 |
| Toluene * 3 | 0.3344 | 30.8 | 15.00 | 92.10 |
| Sodium Hydroxide | 0.0334 | 2.9 | 1.50 | 40.08 |

The mixture of R-[3,5-bis(trifluoromethyl)phenyl]ethan-1-ol (99.9% EE) in toluene was cooled to 5° C. and triethylamine (1.48 equiv) was charged followed by toluene line wash (0.43 equiv). Mesylchloride (1.08 equiv) was charged dropwise maintaining temperature below 15° C. and the line washed with toluene (0.82 equiv). The vessel was heated to 30° C. and held for 1 hour to enable reaction to reach completion. The resulting mixture was cooled to room temperature at which point the triethylamine.HCl may be removed by washing three times, once with water (23.64 equiv.), followed by 10% HCl solution (1.11 equiv) and water (23.64 equiv). The resulting organics were treated with 40% aqueous methylamine (4.98 equiv) at 70° C. at approximately 1.5-2.0 bar for 24 hours. The cooled two-phase reaction mix was separated and the organics washed three times with water (19.23 equiv). The crude free amine was purified by first extracting into aqueous HCl (1.00 equiv) and impurities removed via back extraction with toluene (15.00 equivs). The HCl salt of the amine was then treated with sodium hydroxide, until pH of greater than 11 was attained, then isolated via extraction into an organic solvent (ethyl acetate, toluene or MTBE, 15.00 equiv) and concentrated under reduced pressure. Following this methodology a high enantiomeric excess could be maintained with a typical drop of 99.9% EE to 99.5% EE. Assays of greater than 97.5% w/w and through yields of greater than 80% were achieved.

What is claimed is:

1. A process for the preparation of a compound of Formula 1: 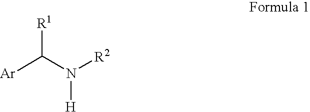
wherein
Ar represents an optionally substituted hydrocarbyl or an optionally substituted heterocyclyl group comprising an aromatic moiety; and
R¹ and R² each independently represent an optionally substituted hydrocarbyl or an optionally substituted heterocyclyl group;
said process comprising:
a) reducing a compound of Formula 2 to form a compound of Formula 3: 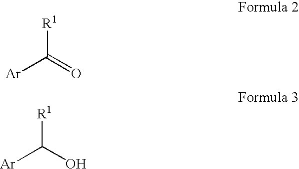
b) activating the compound of Formula 3 to form a compound of Formula 4: 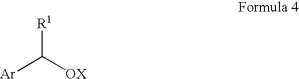
wherein OX represents a leaving group; and
c) coupling the compound of Formula 4 to a compound of Formula 5: 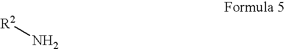
to form a compound of Formula 1.

2. A process according to claim 1 wherein Ar and R¹ are different and a stereoselective reduction system is employed.

3. A process according to claim 2 wherein the stereoselective reduction system is a chiral coordinated transition metal catalysed transfer hydrogenation process or enzymic reduction systems.

4. A process according to claim 3 wherein the chiral coordinated transition metal catalysed transfer hydrogenation process employs a transfer hydrogenation catalyst of formula (a) 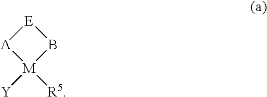
wherein:
R⁵ represents a neutral optionally substituted hydrocarbyl, a neutral optionally substituted perhalogenated hydrocarbyl, or an optionally substituted cyclopentadienyl ligand;
A represents an optionally substituted nitrogen;
B represents an optionally substituted nitrogen, oxygen, sulphur or phosphorous;
E represents a linking group;
M represents a metal capable of catalysing transfer hydrogenation; and
Y represents an anionic group, a basic ligand or a vacant site;
provided that at least one of A or B comprises a substituted nitrogen; and
provided that when Y is not a vacant site that at least one of A or B carries a hydrogen atom.

5. A process according to claim 4 where the transfer hydrogenation catalyst is a transition metal catalyst of Formula B(i-iv) or Formula C(i-viii):

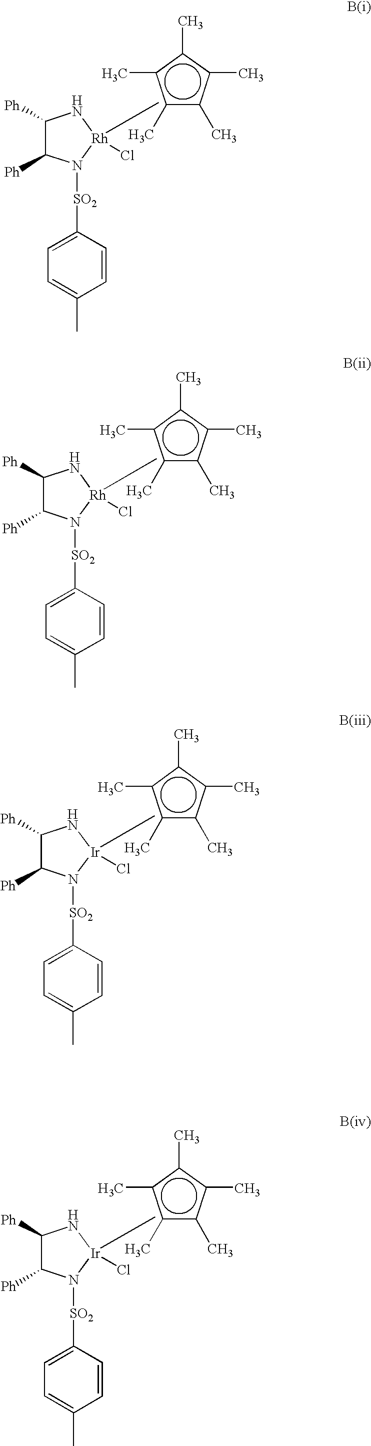

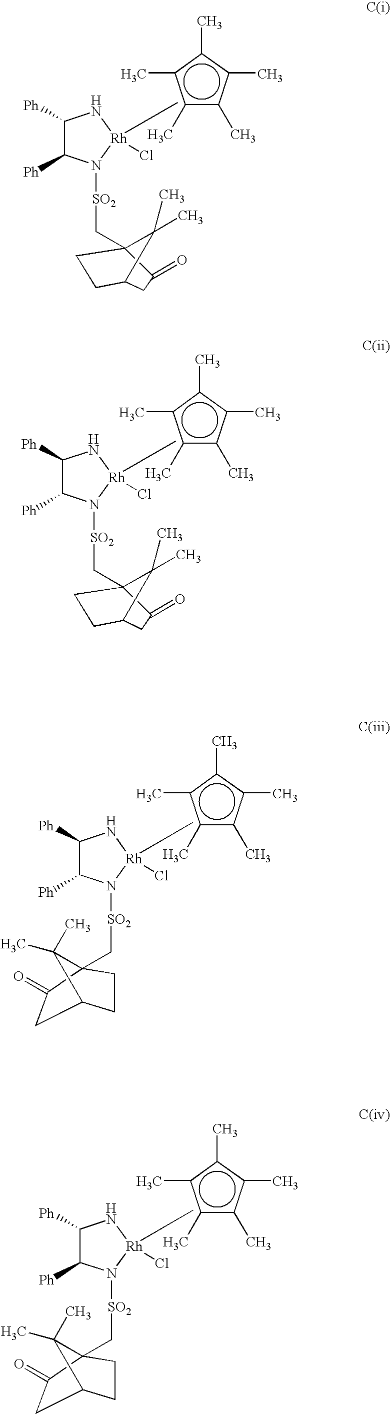

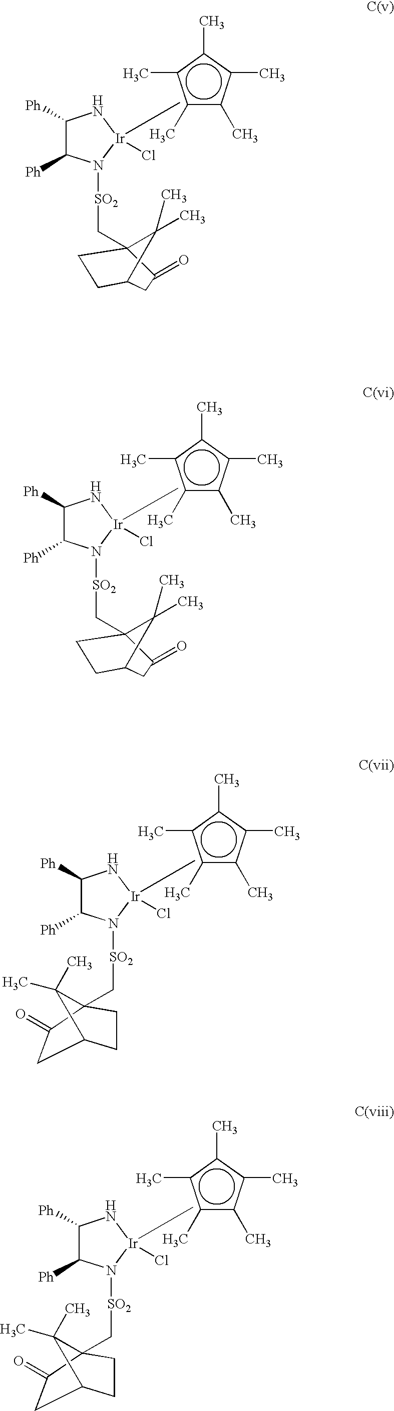

6. A process according to claim 5 where the transfer hydrogenation catalyst is a transition metal catalyst of Formula B(i-iv).

7. A process according to claim 1 wherein the compound of Formula 2 is a compound of Formula 2a: 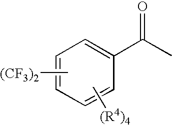
wherein R⁴ each independently represents hydrogen or a substituent group.

8. A process according to claim 7 wherein the compound of Formula 2 is a compound of Formula 2b: 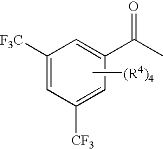
wherein R⁴ each independently represents hydrogen or a substituent group.

9. A process according to claim 7 wherein R⁴ are all hydrogen.

10. A process according to claim 1 wherein X is an acetyl, trifluoroacetyl, methanesulphonyl, trifluoromethylsulphonyl or toluenesulphonyl group.

11. A process according to claim 1 wherein the compound of Formula 1 is obtained in enantiomeric excess.

12. A process for the transfer hydrogenation of a compound of formula (6) to produce a compound of formula (7) 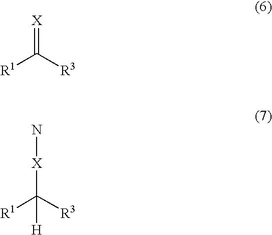
wherein:
X represents O; and
R¹ and R³ each independently represents a hydrogen atom, an optionally substituted hydrocarbyl, a perhalogenated hydrocarbyl or an optionally substituted heterocyclyl group, or R¹ & R³ optionally being linked in such a way as to form an optionally substituted ring(s),
said process comprising reacting the compound of formula (6) with a hydrogen donor in the presence of a transfer hydrogenation catalyst in a multi-phase system.

13. A process according to claim 12 wherein the multi-phase system is a two phase system comprising a liquid water immiscible phase and an aqueous or water phase.

14. A process according to claim 13 wherein the transfer hydrogenation catalyst is soluble in the water immiscible solvent phase and the hydrogen donor is soluble in the aqueous or water phase.

15. A process according to claim 12 wherein a phase transfer catalyst is present.

16. A process according to claim 13 wherein the compound of formula 6 is present as a neat oil.
